(19) United States
(12) Patent Application Publication
(10) Pub. No.: US 2024/0033153 A1
(43) Pub. Date: Feb. 1, 2024

(54) POSTURE HOLDING MAT

(71) Applicants: YAMATO-SANGYO CO., LTD., Nara (JP); ACHILLES CORPORATION, Shinjuku-ku, Tokyo (JP)

(72) Inventors: Koji SHIOTANI, Nara (JP); Hirotake HIRAKAWA, Shinjuku-ku, Tokyo (JP); Masayoshi TACHIBANA, Shinjuku-ku, Tokyo (JP); Kazuhisa YAMADA, Shinjuku-ku, Tokyo (JP); Ryuichi YANAGIYAMA, Shinjuku-ku, Tokyo (JP)

(21) Appl. No.: 18/256,676

(22) PCT Filed: Dec. 2, 2021
(86) PCT No.: PCT/JP2021/044196

§ 371 Date: Jun. 9, 2023

(30) Foreign Application Priority Data
Dec. 10, 2020 (JP) 2020-204661

(51) Int. Cl. A61G 13/12; A61L 31/06

(52) U.S. Cl. CPC A61G 13/126; A61L 31/06

(57) ABSTRACT

A posture holding mat according to the present invention is a mat which has a predetermined thickness to hold a posture, the posture holding mat includes: an elastic foam; and a cuttable line which allows the mat to be cut to a predetermined size and in the cuttable line, cuts penetrating in the direction of the thickness are provided at a predetermined interval. In this way, the posture holding mat can be cut by an operator with fingers without use of a cutting tool such as a cutter, can be bent to be used and can be used for holding the posture of a subject during an operation in orthopedics, surgery or the like.

### TECHNICAL FIELD

The present invention relates to a mat which is preferably used, for example, for holding the posture of a subject during an operation in orthopedics, surgery or the like.

### BACKGROUND ART

In the fields of orthopedics, surgery and the like, when an operation is performed for a long period of time while the posture of a subject is being held steady, a sandbag, independent foam blocks of various shapes and sizes, an operation fixing tool such as a restraint band, a negative pressure fixing tool and the like are used.

For example, Patent Literature 1 discloses a posture fixing mat in which a cushion body with foam heads filled in the interior of each of gas-permeable bags coupled in a longitudinal direction is stored in a gas impermeable bag, after the position of the posture of a subject is confirmed, the gas impermeable bag is deaerated to bring the cushion body into close contact with the gas impermeable bag, the foam beads are fixed within the cushion body and thus the posture of the subject is fixed.

Patent Literature 2 discloses a posture holding tool including: a main body formed with a viscoelastic gel-like material of a predetermined thickness and compressive stress and a film of a predetermined thickness, tensile strength and the like which wraps the viscoelastic gel-like material; and a bottom portion formed of an elastomer which has a predetermined thickness and a hardness higher than that of the viscoelastic gel-like material.

As a commercially available product, a mat made of low-resilience urethane foam called “Soft Nurse” (registered trademark) is used as a posture holding mat for an operation.

### CITATION LIST

Patent Literature 1: Japanese Unexamined Patent Application Publication No. 2019-63416

Patent Literature 2: Japanese Unexamined Patent Application Publication No. 2005-52183

### SUMMARY OF INVENTION

### Technical Problem

However, in principle, the posture holding mats disclosed in Patent Literatures 1 and 2 are to be washed and reused after use, and due to their structures, it is impossible to cut them to a desired size for use. Hence, the place, the posture and the like in which the posture holding mats can be used are limited. Although “Soft Nurse” (registered trademark) is also to be washed and reused after use in principle, it can be cut to a desired size. However, a cutting tool such as a cutter is required for cutting. For this reason, there is a demand for further improvement in the performance of mats used in an operating room and the like in which simple operations and quick responses are required.

The present invention is made in view of the conventional problems as described above, and an object of the present invention is to provide a posture holding mat which can be cut by an operator with fingers without use of a cutting tool such as a cutter, can be bent to be used and can be used for holding the posture of a subject during an operation in orthopedics, surgery or the like.

### Solution to Problem

In order to achieve the object described above, a posture holding mat (hereinafter also simply referred to as the “mat”) according to the present invention is a mat that has a predetermined thickness to hold a posture, the posture holding mat includes: an elastic foam; and a cuttable line that allows the mat to be cut to a predetermined size and in the cuttable line, cuts penetrating in the direction of the thickness are provided at a predetermined interval.

In the posture holding mat configured as described above, the cuttable lines are preferably provided in a grid pattern in a surface perpendicular to the direction of the thickness of the mat.

In the posture holding mat configured as described above, a plurality of projections and depressions are preferably formed alternately in two orthogonal directions in plan view on one surface side and/or on the other surface side in the direction of the thickness of the mat.

In the posture holding mat configured as described above, the ratio of the height of the projection to the thickness of the mat is preferably in a range equal to or greater than 15% and equal to or less than 40%.

In the posture holding mat configured as described above, the length of the cut is preferably equal to or greater than 10 mm.

In the posture holding mat configured as described above, the length of a connection portion between the adjacent cuts is preferably in a range equal to or greater than 1 mm and equal to or less than 8 mm.

In the posture holding mat configured as described above, the foam is preferably polyurethane foam.

In the posture holding mat configured as described above, the density of the foam is preferably equal to or greater than 20 kg/m³.

In the posture holding mat configured as described above, a mark is preferably provided to serve as a cut length indicator when the mat is cut. The mark is preferably a groove that is formed in a side surface of the mat and extends in the direction of the thickness of the mat.

### Advantageous Effects of Invention

The posture holding mat of the present invention can be cut by an operator with fingers without use of a cutting tool such as a cutter and can be bent to be used. In this way, the posture holding mat can be effectively used for holding the posture of a subject during an operation in orthopedics, surgery or the like.

### BRIEF DESCRIPTION OF DRAWINGS

FIG. 1 is a perspective view viewed from the upper side of a mat according to a first embodiment;

FIG. 2 is a perspective view viewed from the lower side of the mat shown in FIG. 1;

FIG. 3 is a vertical cross-sectional view of the mat shown in FIG. 1;

FIG. 4 is a partial enlarged view of a cuttable line L;

FIG. 5 is a perspective view showing an example of use of a mat according to the present invention;

FIG. 6 is a perspective view showing another example of use of the mat according to the present invention; and

FIG. 7 is a bottom view of a mat according to a second embodiment.

### DESCRIPTION OF EMBODIMENTS

Although a mat according to the present invention will be described below with reference to drawings, the present invention is not limited to embodiments at all. In the present specification, the “direction of thickness”, an “X-direction” and a “Y-direction” are assumed to mean the direction of thickness, an X-direction and a Y-direction indicated in the drawings.

### First Embodiment

### Mat

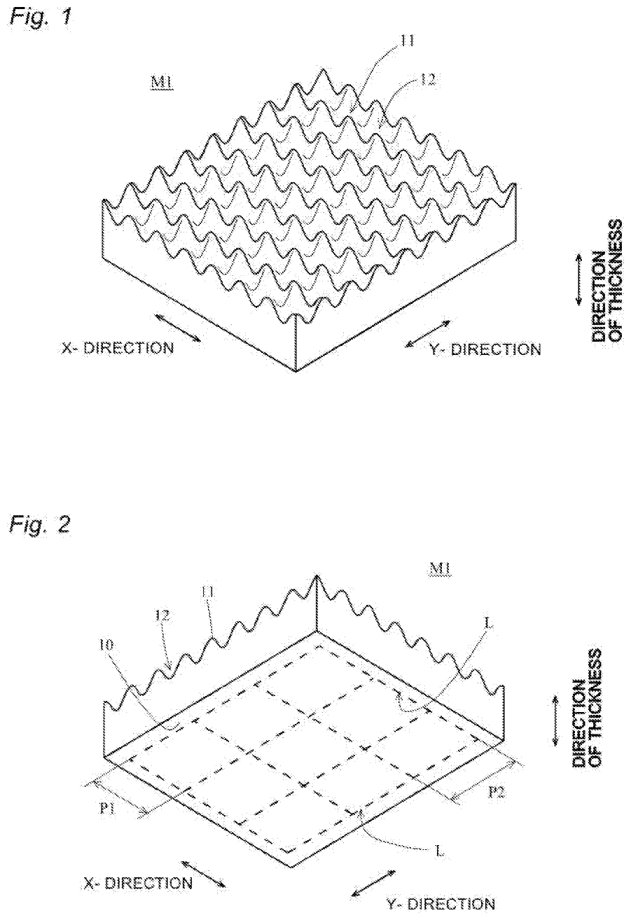

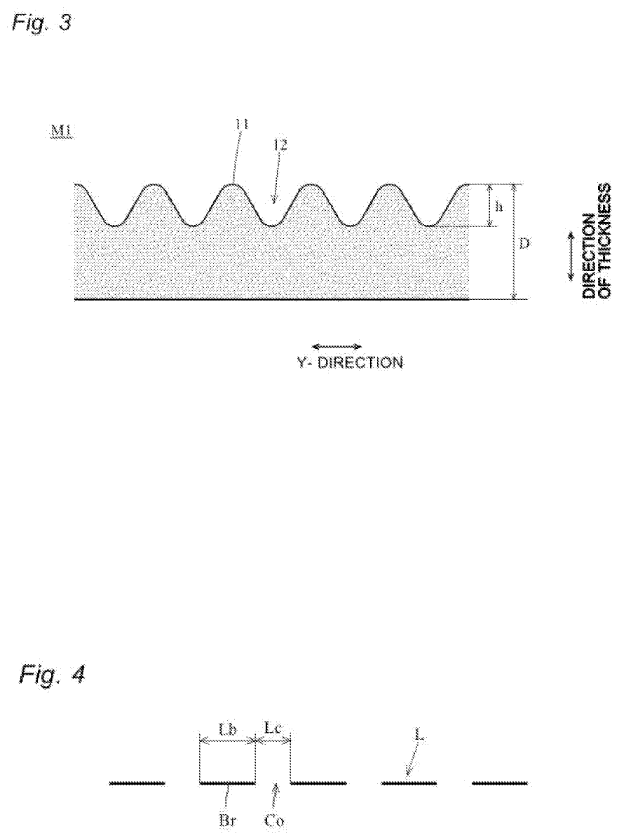

FIG. 1 shows a perspective view viewed from the upper side of a mat M1 according to a first embodiment in an up/down direction which is the direction of thickness, FIG. 2 shows a perspective view viewed from the lower side in the up/down direction and FIG. 3 shows a vertical cross-sectional view. The mat M1 shown in these figures is rectangular in plan view and includes an elastic foam. Profile processing is performed on one surface side (upper surface side in FIG. 1) in the direction of thickness such that a plurality of projections 11 and depressions 12 are formed alternately in two orthogonal directions in plan view. The profile processing as described above is performed to enhance body pressure dispersion and breathability so as to reduce bedsore and the like.

Although the thickness D (shown in FIG. 3) of the mat M1 is not particularly limited, in general, the thickness D is preferably equal to or greater than 40 mm and equal or less than 80 mm, and more preferably equal to or greater than 50 mm and equal or less than 70 mm. Although in terms of enhancing body pressure dispersion, breathability and the like, the height of the projections 11 is considered to be increased, durability and the like are lowered as the height of the projections 11 is increased. Hence, it is generally recommended that the height h of the projections 11 (shown in FIG. 3) he equal to or greater than 15% and equal to or less than 40% of the thickness D of the mat M1 (shown in FIG. 3). The profile processing may be performed not only on the one surface side but also on the other surface side in the direction of thickness of the mat M1 or both the surface sides may be planar without the profile processing being performed thereon. However, when the mat M1 is used, for example, for holding the posture of a subject during an operation in orthopedics, surgery or the like, the profile processing is preferably performed on at least one of the surface sides, and when the mat M1 is used, the mat M1 is preferably arranged such that the side on which the profile processing is performed is the side of the subject.

As the foam of the mat M1, for example, an elastic resin foam such as polyurethane foam, polyethylene foam or polypropylene foam can be preferably used. Among them, polyurethane foam is preferably used in which impact resilience is low and body pressure dispersion is high. Moreover, in terms of achieving the functions of breathability and water permeability, polyurethane foam of a three-dimensional mesh structure without the provision of cell membranes is recommended. An antistatic function (static electricity prevention), antibacterial performance and antifungal performance may be provided to the mat M1 by containing, in the foam, conductive powder such as carbon, an antibacterial agent and an antifungal agent.

The density of the foam of the mat M1 is preferably equal to or greater than 20 kg/m³. If the density of the foam is less than 20 kg/m³, the mat M1 may tear along a cuttable line against the will of a user when the mat M1 is bent. When the mat M1 is compressed to save space during storage and transportation, there is a risk of insufficient recovery from a compressed state during use. The lower limit value of the density of the foam is more preferably 25 kg/m³. On the other hand, although the upper limit value of the density of the foam is not particularly limited, the upper limit value is generally 50 kg/m³.

### Cuttable Line

As shown in FIG. 2, in the mat M1, a plurality of cuttable lines L which allow the mat M1 to be cut to a predetermined size are provided in a grid pattern at predetermined intervals in the X-direction and the Y-direction. In the cuttable lines L, cuts Br (shown in FIG. 4) penetrating in the direction of thickness are formed at predetermined intervals in the direction of the line. As described above, the cuttable lines L are provided in the mat M1, and thus the user utilizes the cuttable lines L to be able to cut the mat M1 to a desired size with fingers without using a cutting tool such as a cutter. Although in the mat M1 described in the present embodiment, an outer peripheral frame portion 10 (shown in FIG. 2) in which the cuttable lines L are not formed in a peripheral edge portion of the mat M1 in an X-Y plane is provided, the outer peripheral frame portion 10 is provided to suppress, for example, the tear of the mat M1 along the cuttable lines L during transportation, storage or the like, and a configuration in which the outer peripheral frame portion 10 is not provided in the mat M1 may be adopted.

One or two or more cuttable lines L may be provided. When two or more cuttable lines L are provided, the formation intervals P1 and P2 thereof (shown in FIG. 2) are not particularly limited, and are preferably determined as necessary from the purpose of use of the mat M1. For example, when the mat M1 is used, for example, for holding the posture of a subject during an operation in orthopedics, surgery or the like, each of the formation intervals P1 and P2 is preferably equal to or greater than 10 mm and equal to or less than 2200 mm. The cuttable lines L are not limited to be linear, and may be, for example, bent or curved.

FIG. 4 shows a partial enlarged view of the cuttable line L. As shown in FIG. 4, in the cuttable line L, a plurality of cuts Br each of which has a length Lb in the direction of the line are provided at predetermined intervals (connection portions Co: length Lc). The cuts Br penetrate the mat M1 in the direction of thickness. In general, the length Lb of the cut Br of the cuttable line L is preferably equal to or greater than 10 mm. Although the upper limit value is not particularly limited, the upper limit value is about 50 mm. If the length Lb of the cut Br is less than 10 mm, when the user cuts the mat M1, the user has difficulty in inserting the fingers into the cuts Br, and thus it is likely that the user cannot start to smoothly cut the mat M1. It is also likely that the fingers are out of the cuttable lines L and thus the mat M1 tears. On the other hand, when the length Lb of the cut Br is greater than 50 mm, the fingers of the user are easily inserted into the cuts Br but unintended separation may occur during transportation or the like.

The length Lc of the connection portion Co which forms the cuttable line L together with the cut Br and is provided between adjacent cuts Br is preferably equal to or greater than 1 mm and equal to or less than 8 mm. When the length Lc of the connection portion Co is less than 1 mm, unintended separation may occur during transportation or the like. On the other hand, when the length Lc of the connection portion Co is greater than 8 mm, the cut surface of the mat M1 may be rough, and chips may be produced. The length Lc of the connection portion Co is more preferably equal to or greater than 1 mm and equal to or less than 6 mm, further preferably equal to or greater than 1 mm and equal to or less than 4 mm and particularly preferably equal to or greater than 1 m and equal to or less than 3 mm.

Although the ratio of the length Lb of the cut Br to the length Lc of the connection portion Co in the cuttable line L is not particularly limited, in general, the length Lb of the cut Br is preferably longer than the length Lc of the connection portion Co.

The connection portion Co preferably connects the cuts Br in at least a part thereof in the direction of thickness, and, for example, a cut which has a predetermined depth in the direction of thickness of the mat M1 may be provided in the connection portion Co. The cut as described above is formed, and thus the mat M1 is more easily cut along the cuttable lines L, and the cutting of the mat M1 out of the cuttable lines L is suppressed.

### Usage Example 1

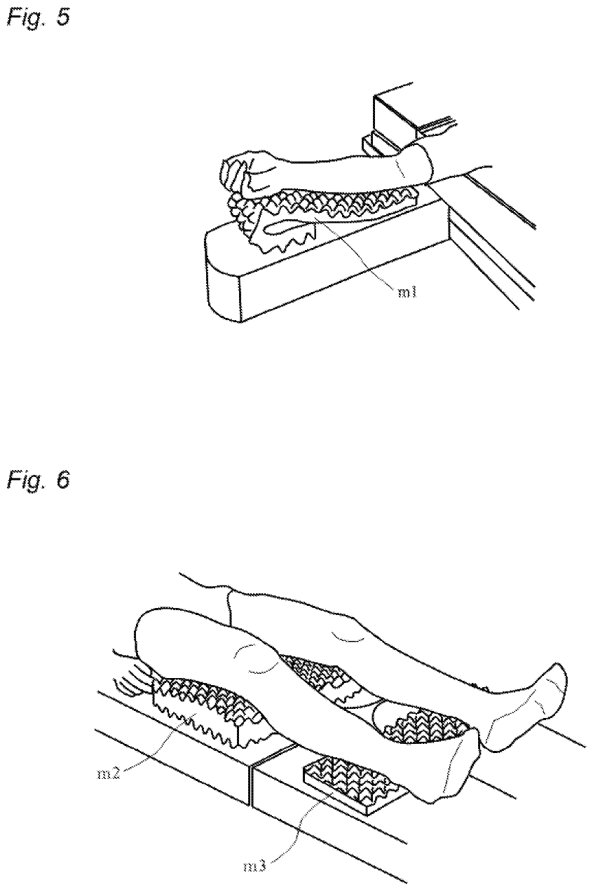

FIG. 5 shows an example of use of the mat of the present invention. In the example of use shown in FIG. 5, the right hand of a subject at a supine position extending vertically from the torso of the subject is held on an operation table with a small mat m1 such that the hand is located slightly above a horizontal plane. Specifically, from an unillustrated main mat (for example, 1900 mm×800 mm) in Which the profile processing is performed on one side surface in the direction of thickness, the small mat m1 is cut which is rectangular in plan view and in which its length in a lateral direction is longer than the longest diameter of the cross section of the right arm and its length in a longitudinal direction is several tens of centimeters longer than the right arm. Then, the entire right arm of the subject is placed on the surface of the small mat m1 on which the profile processing is performed, a tip end of the small mat m1 on the right side in the longitudinal direction is bent downward 180 degrees, the right hand of the subject is placed on a part where two layers are formed by the bending and the right arm is held in a state where the right arm is inclined slightly downward from the side of the hand toward the side of the shoulder.

### Usage Example 2

FIG. 6 shows another example of use of the mat of the present invention. In the example of use shown in FIG. 6, both the legs of a subject in a supine position are held on an operation table with two small mats m2 and m3 such that the knees of the legs are slightly bent. Specifically, from an unillustrated main mat in which the profile processing is performed on both surfaces in the direction of thickness, the small mat m2 is cut which is rectangular in plan view and in which its length in a lateral direction is substantially equal to the length of the femurs in the direction of a bone and its length in a longitudinal direction is longer than the width of the subject, and the small mat m2 is arranged between the femurs and the operation table. From an unillustrated main mat in which the profile processing is performed on one surface side in the direction of thickness, the small mat m3 is cut which is rectangular in plan view and in which its length in a lateral direction is shorter than the length of the lower legs in the direction of a bone and its length in a longitudinal direction is longer than e width of the subject, and the small mat m3 is arranged between the ankle side of the lower legs and the operation table. The thickness of the small mat m2 is greater than that of the small mat m3, and thus both the legs of the subject are held on the operation table such that the knees are slightly bent.

As described above, in the mat of the present invention, the cuttable lines are used, and thus a small mat having a desired size can be cut from the main mat, and the mat can be bent 180 degrees to be used. Moreover, after use, the mat of the present invention can be discarded without being washed and reused like a conventional one.

### Second Embodiment

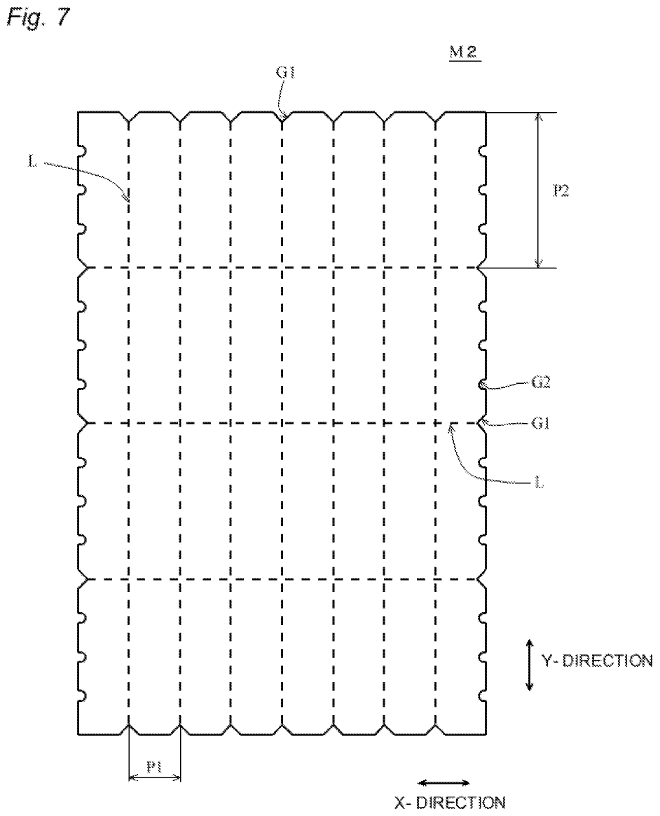

FIG. 7 shows a bottom view of a mat M2 according to a second embodiment. The mat M2 shown in FIG. 7 differs from the mat M1 of the first embodiment in that the outer peripheral frame portion 10 in which the cuttable lines L are not formed is not provided in the peripheral edge portion of the mat M2 and grooves which are marks serving as a cut length indicator when the mat is cut and extend in the direction of thickness of the mat are provided in the side surfaces of the mat M2. Since the other configurations are the same as those in the mat M1 of the first embodiment except the shape and the size of the mat, the same parts as in the mat M1 are identified with the same reference signs, and the description thereof are omitted here.

The mat M2 includes seven cuttable lines L which extend linearly in the Y-direction and are formed at intervals P1 and three cuttable lines L which extend linearly in the X-direction and are formed at intervals P2. In each of the side surfaces of the mat M2 reached by these cuttable lines L, V-shaped grooves (hereinafter also referred to as the “V-grooves”) G1 which extend in the direction of thickness of the mat M2 (direction perpendicular to the plane of FIG. 7) are formed. More specifically, the V-grooves are formed such that the ends of the cuttable lines L abut against the bottom tops of the V-grooves G1.

The V-grooves G1 are formed at the ends of the cuttable lines L, and thus it is easy to find the formation positions of the cuttable lines L which are difficult to visually recognize in the mat M2 in a normal state where an external force such as bending stress is not applied, and the mat M2 can be more easily separated with the V-groove used as a separation starting point by applying a force with fingers so as to push and spread the side surface opposite the V-groove.

In the mat M2, U-shaped grooves (hereinafter also referred to as the “U-grooves”) G2 are formed between V-grooves G1 adjacent in the Y-direction at predetermined intervals. A plurality of U-grooves G2 are formed at regular intervals between the V-grooves G1, and thus the mat of a desired size can easily be grasped so as to be cut. Specifically, even when it is necessary to cut the mat of a length other than an integral multiple of the formation interval P2 of the cuttable lines L, the U-groove G2 is used as an indicator, and thus it is possible to easily determine the position at which the mat is cut. For example, when the length between the V-groove G-1 and the U-groove G2 and the length between the U-groove G2 and the U-groove G2 each are assumed to be 5 cm, and a met having a length of 25 cm in the V-direction is cut, since the length from a predetermined V-groove G1 to the subsequent V-groove G1 is 20 cm, the mat is preferably cut at the position of the U-groove G2 which is adjacent to the V-groove G1 so as to be distant therefrom.

The shape of the groove formed in the mat M2 is not limited to the V shape and the U shape, and a conventionally known shape can be adopted. As the shape of the groove, one type may be provided or two or more types may be provided. However, in order to be able to determine whether or not positions are the formation positions of the cuttable lines L, it is preferable to provide two or more types of shapes of the grooves and to provide a different shape of the groove at each of the formation position of the cuttable line L and the position other than the formation position.

Although in the present embodiment, the V-grooves G1 and the U-grooves G2 formed in the side surfaces of the mat M2 are used as a cut length indicator when the mat is cut, the mark for the cut length of the mat is not limited to the groove, and may be, for example, a print display on the surface of the mat M2 or the like.

### EXAMPLES

### Examples 1 to 6

In polyurethane foam which had a length of 150 mm in the X-direction, a length of 200 mm in the Y-direction, a thickness of 60 mm and a density of 25 kg/m³ and in which the profile processing (projections of 20 mm) was performed on one surface side, cuttable lines parallel to the X-direction or the Y-direction. were formed at intervals of 50 mm in the X-direction and at intervals of 75 mm in the Y-direction, and thus mats in Examples 1 to 6 were produced. The lengths of cuts Br and connection portions Co of the cuttable lines were as shown in Table 1. Then, on the produced mats, evaluation tests of the following items were performed. Evaluations results are shown in Table 1.

### Examples 7 to 11

Mats were produced and evaluation tests were performed as in Examples 2 to 6 except that the height of projections 11 on which the profile processing was performed was 10 mm. Evaluations results are shown in Table 1.

### Bending Evaluation

The mat was bent 180 degrees with a center portion in the X-direction being the center, was compressed for 2 to 3 seconds and thereafter whether or not a crack occurs in the cuttable lines was checked. Likewise, the mat was bent 180 degrees with a center portion in the Y-direction being the center, was compressed for 2 to 3 seconds and thereafter whether or not a crack occurs in the cuttable lines was checked.

### Ease of Starting to Cut

Fingers were inserted into the cuts of the mat to push and spread the cuts, and ease of starting to cut was evaluated based on the following criteria.

Excellent: The first connection portion was able to be easily cut with the fingers inserted into the cuts.

Good: The first connection portion was able to be cut with the fingers inserted into the cuts but some force was required.

Poor: A force was required when the first connection portion was cut with the fingers inserted into the cuts, and it was difficult to cut the mat.

### Ease of Cutting

Ease of pushing, spreading and developing the cuts of the mat was evaluated based on the following criteria.

Excellent: The cuts were able to be easily pushed, spread and developed.

Good: Some force was required to push and spread the cuts but the cuts were able to be developed.

Poor: A force was required to push and spread the cuts and it was difficult to develop the cuts.

### Occurrence of Cut other than Connection Portion

Whether or not a cut in portions other the connection portions occurs when the cuts were developed was evaluated based on the following criteria.

Excellent: The connection portions were easily cut, and were easily cut linearly along the cuts.

Good: Some force was required for cutting the connection portions, and care was needed to be taken to cut the connection portions linearly along the cuts.

Poor: A force was required to cut the connection portions, and considerable care was needed to be taken to cut the connection portions linearly along the cuts.

### State of Cut Surface and Chips

The state of the cut surface and whether or not chips were produced after the mat was cut along the cuttable lines were evaluated based on the following criteria.

Excellent: The cut surface was smooth, and no chips were produced.

Good: Small depressions and projections were present but no chips were produced.

Poor: Depressions and projections were remarkable, the cut surface was rough and chips might be produced.

TABLE 1

| | Profile | | | | Cutting evaluation | | | |
|---|---|---|---|---|---|---|---|---|
| | processing | | | | | | Occurrence | |
| | Thickness/ | Cuttable line | | | Ease of | | of cut other | State of cut |
| | Projection | Cut | Connection | Bending | starting | Ease of | than connection | surface and |
| | (mm) | (mm) | Portion | evaluation | to cut | cutting | portion | chips |
| Example 1 | 60/20 | 12 | 1 | No cracks | Excellent | Excellent | Excellent | Excellent |
| Example 2 | | 12 | 2 | No cracks | Excellent | Excellent | Excellent | Excellent |
| Example 3 | | 12 | 3 | No cracks | Good~Excellent | Good~Excellent | Good | Good |
| Example 4 | | 12 | 4 | No cracks | Good | Good | Good | Poor |
| Example 5 | | 12 | 6 | No cracks | Good | Poor | Good | Poor |
| Example 6 | | 16 | 8 | No cracks | Poor | Poor | Good | Poor |
| Example 7 | 60/10 | 12 | 2 | No cracks | Excellent | Excellent | Excellent | Excellent |
| Example 8 | | 12 | 3 | No cracks | Good~Excellent | Good~Excellent | Good | Good |
| Example 9 | | 12 | 4 | No cracks | Good | Good | Good | Poor |
| Example 10 | | 12 | 6 | No cracks | Good | Poor | Good | Poor |
| Example 11 | | 16 | 8 | No cracks | Poor | Poor | Good | Poor |

As is clear from Table 1, it was found that among the mats in which the height of projections subjected to the profile processing was 20 mm, the mats of Examples 1 and 2 in which the length of the cut in the cuttable line was 12 mm and the lengths of the connection portions were respectively 1 mm and 2 mm were excellent both in the bending evaluation and the cutting evaluation. It was found that among the mats in which the height of projections subjected to the profile processing was 10 the mat of Example 7 in which the length of the cut in the cuttable line was 12 mm and the length of the connection portions was 2 mm was excellent both in the bending evaluation and the cutting evaluation.

It was found that in the mats of Examples 3 to 5 and Examples 8 to 10 in which the length of the cut in the cuttable line was 12 mm and the lengths of the connection portions were 3 mm, 4 mm and 6 mm, the evaluation items of the “ease of starting to cut”, the “ease of cutting” and the “state of cut surface and chips” tended to be lowered as the length of the connection portions was increased.

It was found that in the mats of Examples 6 and 11 in which the length of the cut in the cuttable line was 16 mm, the length of the connection portions was 8 mm, the ratio of the length of the cut to the length of the connection portions was the same as that in the mats of Examples 5 and 10 and the lengths are increased, as compared with the mats of Examples 5 and 10, the “ease of starting to cut” was lowered but the other evaluation items were the same.

### INDUSTRIAL APPLICABILITY

The mat of the present invention can be cut by an operator with lingers without use of a cutting tool such as a cutler, can be bent to be used and can be effectively used for holding the posture of a subject during an operation in orthopedics, surgery or the like.

### REFERENCE SIGNS LIST

D thickness of mat
h height of projection
L cuttable line
M1, M2 mat
m1 small mat
m2 small mat
m3 small mat
10 outer peripheral frame portion
11 projection
12 depression
Br cut
Co connection portion
G1 V-groove
G2 U-groove
Lb length of cut
Lc length of connection portion

1. A posture holding mat that has a predetermined thickness to hold a posture, the posture holding mat comprising:
an elastic foam; and
a cuttable line that allows the mat to be cut to a predetermined size,
wherein in the cuttable line, cuts penetrating in a direction of the thickness are provided at a predetermined interval.

2. The posture holding mat according to claim 1,
wherein the cuttable lines are provided in a grid pattern in a surface perpendicular to the direction of the thickness of the mat.

3. The posture holding mat according to claim 1,
wherein a plurality of projections and depressions are formed alternately in two orthogonal directions in plan view on one surface side and/or on the other surface side in the direction of the thickness of the mat.

4. The posture holding mat according to claim 3,
wherein a ratio of a height of the projection to the thickness of the mat is in a range equal to or greater than 15% and equal to or less than 40%.

5. The posture holding mat according to claim 1,
wherein a length of the cut is equal to or greater than 10 mm.

6. The posture holding mat according to claim 1,
wherein a length of a connection portion between the adjacent cuts is in a range equal to or greater than 1 mm and equal to or less than 8 mm.

7. The posture holding mat according to claim 1,
wherein the foam is polyurethane foam.

8. The posture holding mat according to claim 1,
wherein a density of the foam is equal to or greater than 20 kg/m³.

9. The posture holding mat according to claim 1,
wherein a mark is provided to serve as a cut length indicator when the mat is cut.

10. The posture holding mat according to claim 9,
wherein the mark is a groove that is formed in a side surface of the mat and extends in the direction of the thickness of the mat.
